(19) United States
(12) Patent Application Publication
(10) Pub. No.: US 2006/0264676 A1
(43) Pub. Date: Nov. 23, 2006

(54) METHOD FOR COATING METAL SURFACES WITH CORROSION INHIBITING POLYMER LAYERS

(75) Inventors: Marc L.A.D. Mertens, Oss (NL); Rene van Schaik, Eindhoven (NL)

Correspondence Address: SENNIGER POWERS, ONE METROPOLITAN SQUARE, 16TH FLOOR, ST LOUIS, MO, 63102, US

(73) Assignee: Enthone Inc., West Haven, CT (US)

(21) Appl. No.: 11/279,696

(22) Filed: Apr. 13, 2006

(30) Foreign Application Priority Data
Apr. 13, 2005 (EP) 05008022.5

(51) Int. Cl. C07C 45/00

(52) U.S. Cl. 568/454

(57) ABSTRACT

A method for coating a metal having a metal surface layer having an oxide layer thereon with a corrosion-inhibiting polymer resin layer. The method comprises contacting the surface of the metal with a pretreatment composition comprising an organophosphorus compound comprising phosphorus having an oxidation state of +3 and an alkyl group capable of interacting with a plastic monomer resin or plastic polymer resin; and contacting the pretreated metal surface with a sealant composition comprising the plastic monomeric and/or polymeric resin.

### FIELD OF THE INVENTION

The present invention relates to a method for coating metal surfaces with corrosion-inhibiting polymer layers.

### BACKGROUND OF THE INVENTION

Known methods for protecting metal surfaces from corrosion involve deposition of what is known as a conversion layer, which is a chromate material precipitated from an electrolytic solution containing chromium(III) and chromium(VI) salts, phosphate ions, and fluoride ions. The chromate conversion coating provides a rough surface which serves as an adhesion layer for adhering a protective polymer layer.

Environmental concerns, including the expenses involved in treating waste water containing hexavalent chromium ions, motivated the search for an adhesion layer which avoids the use of chromium. Alcoa is the assignee of several patents (see U.S. Pat. Nos. 6,030,710; 6,696,106; and 6,020,030) which disclose primer coatings comprising organophosphorus compounds which served as an adhesive layer between a metal surface and a protective polymer layer. The patents discuss their compounds in the context of protecting aluminum surfaces.

Accordingly, a need continues to exist for a primer composition which applies a strongly adhering layer between a metal surface and a protective polymer layer which avoids the use of chromate conversion coatings.

### SUMMARY OF THE INVENTION

Among the various aspects of the present invention may be noted a process for coating a metal surface with a protective polymer layer, which inhibits corrosion of the metal surface.

Briefly, therefore, the present invention is directed to a method for coating a metal having a metal surface layer having an oxide layer thereon with a corrosion-inhibiting polymer resin layer, the method comprising contacting the surface of the metal with a pretreatment composition to pretreat the metal surface, the pretreatment composition comprising an organophosphorus compound comprising phosphorus having an oxidation state of +3 and an alkyl group capable of interacting with a plastic monomer resin or plastic polymer resin; and contacting the primed metal surface with a sealant composition comprising said plastic monomeric resin, said plastic polymeric resin, or a combination thereof.

Other objects and features of the invention will be in part apparent and in part pointed out hereinafter.

### DETAILED DESCRIPTION OF THE PREFERRED EMBODIMENT(S)

In accordance with the present invention, a metal substrate is coated with a corrosion-inhibiting monomeric or polymeric resin which is applied on top of a pretreatment coating formed from an organophosphorus compound. Organophosphorus compounds, especially those containing phosphorus in its 3+ oxidation state are capable of interacting with and chemically bonding to the surface of a metal substrate, particularly when the metal substrate comprises a surface oxide layer. Accordingly, the application of an organophosphorus compound to a metal surface having a surface oxide layer thereon is effective to form a corrosion-inhibiting layer on the metal surface.

The process of the present invention employs the organophosphorus compound undercoating as a pretreatment layer for the additional application of a monomeric or polymeric resin layer which interacts with an organic moiety present in the organophosphorus compound. In other words, the organophosphorus compound comprises a phosphorus moiety which chemically bonds to the metal oxide surface of a metal substrate and additionally comprises an organic moiety capable of interacting with a monomeric and polymeric resin. The pretreatment coating formed by the organophosphorus compound is capable of acting as a strongly adhesive layer between the metal surface and the monomeric and polymeric resin.

The pretreatment composition of the present invention comprises an organophosphorus compound. The organophosphorus compound can be a compound comprising an alkene moiety and a phosphorus moiety, can be a polymer comprising a monomer derived from a compound comprising an alkene moiety and a phosphorus-containing moiety, or can be a combination thereof. The phosphorus moiety in the organophosphorus compound chemically reacts with oxide layer present on the surface of the metal substrate to form a pretreatment coating over the metal substrate. The metal substrate typically has a passivating metal oxide layer. The phosphorus moiety of the organophosphorus compound contains phosphorus in its 3+ oxidation state, which is capable of forming chemical bonds with surface oxide present on the metal surface. Metal oxide on the surface reacts with phosphonate compounds to form a chemical bond between the surface metal oxide and phosphorus. The reaction between an exemplary surface metal oxide, such as zinc oxide, and an alkenyl phosphonate occurs as shown:
ZnOₓ₍ₛ₎+R—PO₃⁻_{(aq)}=>Zn—O—PO₂—R

Each phosphonate having the general structure shown in the above reaction can react with one, two, or three oxygen atoms on the surface of the surface metal layer. The reaction causes the phosphorus oxide compound to be chemically bonded to the surface metal oxide.

Exemplary organophosphorus compounds comprising an alkene moiety and a phosphorus moiety include alkenyl phosphonic acids, alkenyl phosphonate salts, and alkenyl phosphonate esters. Alkenyl phosphonic acids, alkenyl phosphonate salts, and alkenyl phosphonate esters compounds can have the following structure:

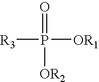

wherein R₁ and R₂ are each independently hydrogen, a counter cation, or a substituted or unsubstituted alkyl group, and R₃ is a substituted or unsubstituted alkenyl group. Exemplary counter cations in the above formula include sodium ions, potassium ions, and ammonium ions.

Preferably, the alkenyl phosphonate compound is vinyl phosphonic acid, its salt, or its ester and has the following structure:

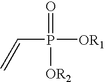

wherein R₁ and R₂ are each independently hydrogen, a counter cation, or a substituted or unsubstituted alkyl group. Vinyl phosphonic acid has the structure:

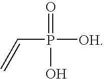

The organophosphorus compound can also be a polymer comprising a monomer derived from a compound comprising an alkene moiety and a phosphorus moiety wherein phosphorus in the phosphorus moiety has an oxidation state of +3. The above-described alkenyl phosphonic acids, alkenyl phosphonate salts, and alkenyl phosphonate esters contain the ethene moiety (C═C) and are thus polymerizable. Accordingly, the organophosphorus compound of the present invention can be a polymer resulting from the polymerization of alkenyl phosphonic acids, alkenyl phosphonate salts, and alkenyl phosphonate esters. The ethene moiety thus forms an alkyl polymer backbone. Polymerization is initiated by an initiating moiety

Where the only monomer present is an alkenyl phosphonic acid, alkenyl phosphonate salt, and/or alkenyl phosphonate ester, the polymer can have the structure:

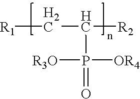

wherein R₁ and R₂ are either an initiating moiety selected from the group consisting of hydrogen, alkyl, ethoxyalkyl, propoxy alkyl, and hydroxyl or a terminating moiety, such that when R₁ is the initiating moiety, R₂ is the terminating moiety and when R₂ is the initiating moiety, R₁ is the terminating moiety; R₃ and R₄ are each independently hydrogen, a counter cation, or a substituted or unsubstituted alkyl group. In a preferred polymer, R₁, R₂, R₃, and R₄ are all hydrogen, and the polymer has the following structure:

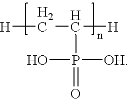

In this polymer, the value of n is such that the polymer can have a molecular weight between about 25,000 g/mol and about 100,000 g/mol.

The polymer can also include a second monomer which co-polymerizes with the alkenyl phosphonic acids, alkenyl phosphonate salts, and alkenyl phosphonate esters. The polymer can be arranged in block, random, or alternating configuration.

Preferably, the second monomer has a carboxylate moiety, and the polymer can have the structure:

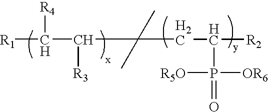

wherein R₁ and R₂ are either an initiating moiety selected from the group consisting of hydrogen, alkyl, ethoxyalkyl, propoxy alkyl, and hydroxyl or a terminating moiety, such that when R₁ is the initiating moiety, R₂ is the terminating moiety and when R₂ is the initiating moiety, R₁ is the terminating moiety; R₃ and R₄ are each independently hydrogen, a carboxylate moiety, or an alkyl carboxylate moiety, and at least one of R₃ and R₄ comprises a carboxylate moiety; R₅ and R₆ are each independently hydrogen, a counter cation, or a substituted or unsubstituted alkyl group.

An exemplary co-polymer of the above structure is a copolymer of vinyl phosponic acid and acrylic acid and has the structure:

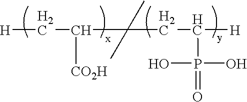

In this copolymer, the ratio of x to y is preferably about 7:3 such that 70% of the repeat units are derived from acrylic acid and 30% of the repeat units are derived from vinylphosphonic aicd. The polymer can have a molecular weight between about 25,000 g/mol and about 100,000 g/mole, such as about 40,000 g/mol.

Another exemplary co-polymer of the above structure is a copolymer of vinyl phosponic acid and fumaric acid and has the structure:

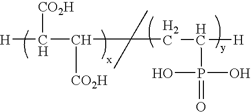

In this copolymer, x and y represent the relative ratio of fumaric acid and vinyl phosphonic acid repeat units. The polymer can have a molecular weight between about 25,000 g/mol and about 100,000 g/mole, such as about 40,000 g/mol.

The sealant composition of the present invention comprises a monomeric and/or a polymeric resin. Exemplary resins applicable for forming a protective polymer layer over the pretreatment coating deposited by the pretreatment composition include polyethylene wax, polyacrylate, polyamine, polyamide, urethane, polyurethane, polyether, polyester, polysilicate, and combinations thereof. As stated above, the organophosphorus compound comprises an organic moiety, which may be an alkenyl group or an alkyl group derived from the polymerization of the alkenyl group. The alkyl group can be derivatized with free carboxylate moieties. Thus, the organophosphorus compound can comprise reactive free alkenyl groups and reactive free carboxylate groups. These free reactive groups are capable of interacting with and bonding to the monomeric resin and polymeric resin materials described above. Accordingly, the organophosphorus compounds, which are chemically bonded to the metal surface layer through P—O bonds and chemically bonded to the resins through carboxylate or alkenyl groups, act as an adhesive layer between the resin and the metal surface layer.

In practicing the method of the present invention, a metal substrate, which has been plated with a zinc layer, is coated with a protective polymer layer. Accordingly, the process involves the following steps:

Pretreating the surface

Rinsing

Electrolytic zinc plating from an alkaline electrolytic zinc plating bath

Rinsing

Exposure to a pretreatment composition comprising the phosphorus compound as described herein

Drying

Exposure to a sealant composition comprising the monomeric and/or polymeric resin as described herein

Drying.

The following examples further illustrate the present invention.

### EXAMPLE 1

### Pretreatment Composition Comprising a Co-Polymer of Acrylic Acid and Vinyl Phosphonic Acid

A pretreatment composition was prepared by adding an organophosphorus compound having the following structure to an aqueous solution:

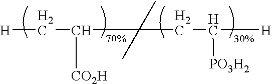

The acrylic acid/vinyl phosphonic acid co-polymer had a molecular weight of approximately 40,000 g/mol, and the co-polymer was added in a concentration of 2% wt. by vol. The pH of the pretreatment composition was adjusted to about 2 using sodium hydroxide and phosphonic acid.

### EXAMPLE 2

### Sealant Composition Comprising Polyethylene Wax

A sealant composition was prepared by adding polyethylene wax (10% solids) using anionic tensides to emulsify and solubilize the polyethylene wax. The pH of the composition was adjusted to about 9.2 using sodium hydroxide and acetic acid.

### EXAMPLE 3

### Corrosion-Protection of Zinc Metal Plated Substrate

The composition of Example 1 and the sealant composition of Example 2 were used to coat a zinc-coated metal substrate with a corrosion-inhibiting polymer resin layer.

The metal substrate was plated with a zinc layer (10 micron average thickness) using Enthobrite® NCZ Dimension, available from Enthone Inc. (West Haven, Conn.) according to the datasheet conditions.

After a cascade water rinse (twice, one minute each time), the zinc-plated substrate was immersed in the pretreatment composition of Example 1 at room temperature for 30 seconds with mild agitation.

The zinc-plated substrate with a pretreatment coating thereon was dried in an oven (10 minutes, 80° C.) and then immersed in the sealant solution of Example 2 at room temperature for 30 seconds.

The zinc-plated substrate having a polymeric layer coating thereon was dried in an oven (10 minutes, 80° C.).

In view of the above, it will be seen that the several objects of the invention are achieved and other advantageous results attained.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above without departing from the scope of the invention, it is intended that all matter contained in the above description and shown in the accompanying drawings shall be interpreted as illustrative and not in a limiting sense. The scope of invention is defined by the appended claims and modifications to the embodiments above may be made that do not depart from the scope of the invention.

What is claimed is:

1. A method for coating a metal having a metal surface layer having an oxide layer thereon with a corrosion-inhibiting polymer resin layer, the method comprising:
contacting the surface of the metal with a pretreatment composition to pretreat the metal surface, the pretreatment composition comprising an organophosphorus compound comprising phosphorus having an oxidation state of +3 and an alkyl group capable of interacting with a plastic monomer resin or plastic polymer resin; and
contacting the pretreated metal surface with a sealant composition comprising said plastic monomeric resin, said plastic polymeric resin, or a combination thereof.

2. The method of claim 1 wherein the organophosphorus compound is a phosphonic acid derivative.

3. The method of claim 1 wherein said alkyl group is a vinyl group.

4. The method of claim 2 wherein said alkyl group is a vinyl group.

5. The method of claim 1 wherein the organophosphorus compound is vinyl phosphinic acid.

6. The method of claim 1 wherein the monomeric resin and/or polymeric resin is selected from the group consisting of polyethylene wax, polyacrylate, polyamine, polyamide, urethane, polyurethane, polyether, polyester, polysilicate, and combinations thereof.

7. The method of claim 2 wherein the monomeric resin and/or polymeric resin is selected from the group consisting of polyethylene wax, polyacrylate, polyamine, polyamide, urethane, polyurethane, polyether, polyester, polysilicate, and combinations thereof.

8. The method of claim 3 wherein the monomeric resin and/or polymeric resin is selected from the group consisting of polyethylene wax, polyacrylate, polyamine, polyamide, urethane, polyurethane, polyether, polyester, polysilicate, and combinations thereof.

9. The method of claim 4 wherein the monomeric resin and/or polymeric resin is selected from the group consisting of polyethylene wax, polyacrylate, polyamine, polyamide, urethane, polyurethane, polyether, polyester, polysilicate, and combinations thereof.

10. The method of claim 5 wherein the monomeric resin and/or polymeric resin is selected from the group consisting of polyethylene wax, polyacrylate, polyamine, polyamide, urethane, polyurethane, polyether, polyester, polysilicate, and combinations thereof.
